(19) United States
(12) Patent Application Publication
(10) Pub. No.: US 2008/0113003 A1
(43) Pub. Date: May 15, 2008

(54) Sinapic Acid Supplementation

(75) Inventors: Henry L. Classen, Saskatoon (CA); Hongyu Qiao, Nan Xiang (CN)

Correspondence Address: BORDEN LADNER GERVAIS LLP;Gail C. Silver, 1100-100 QUEEN ST, OTTAWA, ON, K1P 1J9, omitted

(73) Assignee: UNIVERSITY OF SASKATCHEWAN, Saskatoon, SK (CA)

(21) Appl. No.: 10/599,745

(22) PCT Filed: Apr. 6, 2005
(86) PCT No.: PCT/CA05/00517

§ 371 Date: Oct. 9, 2007

Related U.S. Application Data
(60) Provisional application No. 60/559,439, filed on Apr. 6, 2004.

(51) Int. Cl. A61K 31/192

(52) U.S. Cl. 424/439; 514/570

(57) ABSTRACT

An animal feed composition for monogastric non-human animals is disclosed comprising supplemental sinapic acid or derivatives thereof. Sinapic acid may be supplemented at a level of from 0.0005% to 3% of the feed composition by weight. The feed composition has the beneficial effect of promoting favourable intestinal microbial ecology by reducing pathogenic microbial populations, increasing favourable microbial populations, and increasing performance and growth characteristics in livestock animals.

### CROSS REFERENCE TO RELATED APPLICATIONS

This application claims priority to and all benefit of U.S. Provisional Application No. 60/559,439, filed Apr. 6, 2004, the entirety of which is incorporated herein by reference.

### FIELD OF THE INVENTION

The present invention relates generally to a dietary supplement.

### BACKGROUND OF THE INVENTION

Hydroxycinnamic acid and derivatives are referred to as a group of the secondary phenol metabolites derived from the phenylpropanoid pathway in the plant kingdom. Among the most widely distributed hydroxycinnamic acids in plant tissues are coumaric, caffeic, ferulic, and sinapic acids. These acids can be found in various conjugated forms resulting from enzymatic hydroxylation, O-methylation, O-glycosylation or esterification. They are found both covalently attached to the plant cell wall and as soluble forms in the cytoplasm. Sinapic acid (4-hydroxy-3,5dimethyoxy-cinnamic acid) is a free phenolic acid. Sinapine, a choline-bound esterified form of sinapic acid, is found in many Cruciferous plants with large amounts found in the Brassica family.

Sinapic acid, its derivatives, and sinapine, the form esterified with choline, have traditionally been considered anti-nutritional factors in animal nutrition. Sinapine and sinapic acid are thought to be responsible for the dark color, bitter and sour taste, and astringency or phenol-like flavor of rapeseed meal and therefore may affect palatability of feed having a high proportion of rapeseed meal. Low palatability may reduce feed intake and performance of growing animals, particularly in non-ruminant species. Sinapic acid can bind with protein, such as bovine serum albumin, in vitro and has the potential to bind with proteins and digestive enzymes in vivo. Sinapine has shown the anti-nutritional effect of producing a fishy egg taint in eggs from some strains of laying hens having genetically controlled low levels of trimethylamine oxidative enzymes.

U.S. Pat. No. 6,245,363 teaches methods of treating plant materials with hydrolytic enzymes isolated from Humicola species. Hydrolytic enzymes, such as those isolated from Aspergillus species, are used for hydrolysis of an ester bond of naturally occurring phenolic compounds in plants material, after which the phenolic material can be removed.

U.S. Pat. No. 6, 143,543 teaches the use of Aspergillus or ferulic acid esterase derived therefrom to prepare animal feed. The feed is combined with the microorganism or with the enzyme to allows release of phenolic groups from plant cell wall components within the feed. Canadian Patent Application No. 2,286,694 teaches the use of a phenolic acid esterase for treating plant material to improve digestibility of the plant material in animals.

In an effort to reduce unwanted phenolics in plant material, U.S. Pat. No. 6,501,004 teaches the production of transgenic cruciferous plants having reduced sinapine content. Further, U.S. patent application Ser. No. 340811 (Milkowski et al.), published as US2003/0145354, teaches the reduction of sinapine content in plants by genetic suppression of the enzyme activities of the sinapine biosynthetic pathway.

The view that phenolic esters and acids are anti-nutritive is widespread. Removal or reduction of phenolics by enzymatic and transgenic modification of such plant materials has been undertaken. However, there has been little emphasis is placed on the utilization or supplementation of naturally occurring phenolics into animal feed in such a way that is advantageous to an animal. The microbial ecology of the intestinal tract of livestock is an important component of animal health. Conventional treatment with sub-therapeutic levels of antibiotics may accomplish the effect of lowering levels of unwanted intestinal pathogens, but there is a need for alternatives because of the widespread over-use of antibiotics and the elimination of preventative antibiotic administration in some jurisdictions—Substitute methodologies that do not require antibiotic administration would be of benefit.

### SUMMARY OF THE INVENTION

It is an object of the present invention to obviate or mitigate at least one disadvantage of previous attempts to modify microbial ecology in the intestine, to render animal feed digestible, or to provide a feed product with increased benefit to an animal. Animal nutrition and health can be affected by the gastrointestinal tract microbial community. When conditions permit an optimal microbial environment, improved production can be realized for livestock, and improved health benefits can be realized for both livestock and domestic animals.

In a first aspect, the invention provides an animal feed composition for monogastric non-human animals comprising agriculturally acceptable feed components supplemented with sinapic acid or a derivative thereof.

In a further aspect of the invention, there is provided a nutritional supplement for a monogastric animals comprising isolated sinapic acid or a derivative thereof in combination with an acceptable excipient.

Additionally, according to an aspect of the invention, there is provided a method of promoting favourable microbial ecology in the intestinal tract of a monogastric animal comprising the step of providing the animal with feed supplemented with sinapic acid or a derivative thereof in an amount of from 0.0005% to about 3.0% by weight of feed.

A further aspect of the invention provides a method of improving the nutritional value of an animal feed composition for consumption by monogastric non-human animals comprising the step of supplementing the feed composition with sinapic acid or a derivative thereof.

The invention additionally relates, in another aspect, to a method of reducing short chain fatty acid production in the hind gut of a monogastric animal comprising the step of administering an effective amount of sinapic acid or a derivative thereof to the monogastric animal.

Further, the invention relates, in an additional aspect to a method of increasing short chain fatty acid production in the ileum of a monogastric animal comprising the step of administering an effective amount of sinapic acid or a derivative thereof to the monogastric animal.

In another aspect of the invention, there is provided method of treating or preventing diseases of the intestinal tract arising from growth or colonization of the intestinal tract by pathogenic bacteria, said method comprising the step of administering an effective amount of sinapic acid or a derivative thereof to a monogastric animal in need thereof.

Conventionally, phenolic compounds in animal feed, for example in such components as canola (rapeseed) have been viewed as anti-nutritional factors, and efforts have been made to remove anti-nutritional factors from animal diets. Surprisingly, it was found that supplementation of the diet of monogastric animals with sinapic acid and derivatives thereof benefits the animals by promoting a more favourable microbial ecology within the digestive tract.

As a further benefit of aspects of the invention, improved performance of the animal may be realized, as assessed through such parameters as growth, energy utilization, consumption, and/or feed efficiency. Additionally aspects of the invention in which animal feed is prepared with supplemental sinapic acid or derivatives thereof allows for improved preservation of the feed. Sinapic acid has the additional benefit that it can act as a feed-grade preservative.

Advantageously, sinapic acid can be derived from readily available natural sources such as plants in the Brassica family, for example, canola. Thus, use of sinapic acid and its derivatives according to the invention is economical. Purified or semi-purified sinapic acid may be used, or plant material that has been hydrolysed to form sinapic acid may be added to feed as a non-purified or semi-purified sinapic acid supplement.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figure, wherein:

FIG. 1 is a graphic representation of percentage G+C profiles of cecal microbes upon supplementation of chickens with sinapic acid.

### DETAILED DESCRIPTION

Generally, the present invention provides a dietary supplement. It was surprisingly discovered that supplementation of sinapic acid and its derivatives affect digestive tract microbial ecology. Beneficial bacteria in the gastrointestinal tract (for example Bifidobacterium and Propionibacterium) increase in numbers while detrimental bacteria (such as Escherichia and Salmonella) decrease in numbers. Other evidence of gut microflora modifications are: decreased fermentation in the lower gut, reduced short chain fatty acid production in the cecum, and increased short chain fatty acid production in the ileum. Improved performance of an animal (increased growth, energy utilization, feed consumption and/or feed efficiency) is also a benefit that may be realized according to the invention. The invention is contrary to the conventional belief that sinapic acid and its derivatives should be removed from feed, and act as anti-nutritional factors. Without being limited to theory, the increase in ileal production of short chain fatty acids may be responsible for increased feed digestibility in animals supplemented with sinapic acid.

The term “agriculturally acceptable feed components” refers to those edible, consumable or digestible feed components either currently known in the field of agriculture or which become known to those skilled in the art.

The term “isolated form” when used in reference to sinapic acid or a derivative thereof which is isolated either completely or in part from the plant source from which it is derived. Further, synthetic sinapic acid or derivatives (not requiring isolation from a plant source) are also considered to be in isolated form.

The term “excipient” as used herein refers to any acceptable consumable diluent known in the agricultural art, or which may become known to those of skill in the art. When used in context of a nutritional supplement, the excipient is one that would be considered acceptable for consumption by a monogastric animal.

The term “microbial ecology” refers to the microbes present (either the number and/or the type) within the lumen of the digestive tract, or the conditions within the lumen created or impacted by the microbes present (such as pH or short chain fatty acid content). When used in context of “favourable” microbial ecology, it is to be understood that the desirable characteristics of microbial ecology may differ between applications of the invention. For example, it may be considered “favourable” to increase total microbial content or total short- chain fatty acid production within a certain segment of the intestinal tract. Additionally, it may be favourable to reduce the number or proportion of potential pathogenic bacteria, or increase the number or proportion of acceptable types of bacteria within any segment of the intestinal tract. Any single characteristic, or combination of such characteristics may be considered “favourable” depending on the desired application.

The phrase “improving nutritional value” in context of a feed composition refers to an increase in one or more desirable characteristic of a feed composition. For example, it may be desirable to have a feed composition that results in improved animal performance parameters, such as but not limited to growth, consumption, energy utilization, nutrient utilization, feed efficiency, and/or volatile fatty acid absorption.

The phrase “diseases of the intestinal tract arising from growth or colonization of the intestinal tract by pathogenic bacteria” as used herein refers to any disease or condition, as known to or which may become known to persons skilled in the agricultural or veterinary arts, which is caused, encouraged, or perpetuated by microbial populations or conditions linked to microbial populations that inhabit the lumen of the intestinal tract.

Animals. The invention has potential benefit to all animals, and is not restricted to any particular species. However, of particular advantage is the use of the invention for monogastric animals. For example, poultry, swine, dogs, cats, fish, domestic birds, fur bearing animals (such as foxes and mink), shrimp and other farmed crustaceans, and other monogastric livestock or domestic animals may benefit greatly from the invention.

Preferably, the invention is intended for use in monogastric animals. Aside from livestock and domestic animals, humans may also benefit from the invention, as a preventative nutritional supplement or therapeutic that assists in retaining healthy bowel status.

Source of Sinapic Acid and Derivatives. Sinapic acid and its derivatives may be obtained by purchase of a commercially available source of purified sinapic acid. Alternatively, sinapic acid may be derived from supplementation of the diet with non-purified forms derived from plant material treated to release sinapic acid. For example, plant material high in sinapine (such as canola or other plants in the Brassica family) can be exposed to a hydrolyzation process to produce choline and sinapic acid. The plant material may then be used as is, partially purified to remove certain unwanted materials, or completely purified to isolate the sinapic acid from the remaining material. Plants from which sinapic acid may ultimately be derived include Cruciferae family plants: Brassica napus, Brassica campestris, Brassica rapa, Brassica juncea, and Sinapis alba, and Crambe abyssinica. Other plants that may contain material from which sinapic acid and derivatives thereof may be derived include grains, such as wheat, corn, barley, rye, and oat, and other plants such as sunflower, potatoes, olives, soybean, coffee, grapes, cruciferous vegetables, tobacco and herbs.

Sinapic acid, preferably in a trans-form, can be purchased as a purified compound (for example from Sigma Chemical Co., with a purity of 98.2%), it may be obtained by synthetic derivation from other chemicals, such as ferulic acid, or may be extracted directly from plant materials as free acids or their derivatives as salts, esters (such as sinapine), aldehydes, and alcohols, through any acceptable physical, chemical, and/or biological processing (such as isolation, filtration, evaporation, solvent extraction). As an example, extraction may be done using 78-95% ethanol or combinations with methanol or acetone.

Further, sinapic acid may be prepared from enzymatic or physicochemical hydrolysis/treatment of plant material. For example, enzymes classified in the Enzyme Classification recommendations as E.C.3.1. and subgroups thereof, such as carboxylic acid esterase, ferulic acid esterase, p-coumaric acid esterase, tannase, and phenolic acid esterase, or mild to strong acid or alkaline condition etc. may be uses to treat plant material. Additionally, sinapic acid may be obtained through microbial transformation of sinapine-containing sources, (for example as derived from sinapyl alcohol). Of course, the source of sinapic acid may include compounds prepared from a combination of above means.

Hydrolysis of sinapine to sinapic acid (and choline) can be undertaken by use of a sinapine esterase enzyme system, comprising at least one enzyme having carboxylic ester hydrolase activity, such as sinapine esterase, ferulic acid esterase, p-coumaric acid esterase, tannase, phenolic acid esterase, or other carboxylic esterases. Once processed, the plant material can be added to animal feed as a supplement. The enzymatic process may be conducted using fermentation processes, whereby the enzyme is a component of a microbial system (such as Aspergillus niger), or may be done as a chemical process through exposure to the enzyme without a microbial system. Advantageously, hydrolysis of sinapic acid using a sinapine esterase enzyme system does not promote further metabolism of sinapic acid to biologically active quinines, which may occur if certain other oxidative enzymes are used, such as polyphenol oxidase, monophenol oxygenase, or phenolic acid oxidase.

Enzymatic hydrolysis of sinapine to sinapic acid and choline may be conducted in any manner that would be acceptable to a person skilled in the art. Enzymatic treatment with ferulic acid esterase (FAE) from Aspergillus niger is one of the preferred enzyme classifications with a broad optimal temperature (50-60° C.), and pH range (4.0-6.0) for the effective and efficient hydrolysis of sinapine in water or under citric acid buffer conditions. Use of this enzyme is effective and efficient in the hydrolysis of sinapine both in the in vitro standard sinapine stock solution and in commercial canola meal. After 20 minutes treatment in ether the water or citric acid buffer conditions, sinapine content can be reduced by about 90% in commercial canola meal samples. Such treated plant material can be used to supplement animal feed either in the unpurified state, or can be purified further to concentrate or isolate sinapic acid. Tannase (from Aspergillus) and tyrosinase (for example, from mushroom) are two other examples of enzymes that may be used to break down sinapine to sinapic acid.

In the case where plant material is used and sinapine is hydrolysed to sinapic acid, the choline that is released in the hydrolysis step does not need to be removed before the treated plant material is added to the feed. Choline itself is a vitamin that has nutritional value to animals, and can advantageously be left in the treated plant material and supplemented into a feed mixture. For example, treated canola meal containing sinapic acid and choline as a result of treatment may be used.

The plant material that can be used to obtain sinapic acid includes such material such as seed, leaf, bark, meal or pulp produced by physicochemical processing of plants. Exemplary plants from which sinapic acid may be derived include canola (rapeseed), mustard, cereal grains, such as wheat, corn, barley, rye, and oat, sunflower, potatoes, olives, soybean, coffee, grapes, cruciferous vegetables, tobacco, and herbs. All of these plants may be a source of sinapic acid. Some of these plants yield a meal or residue after physiochemical processing (for example after oil seed extraction) which is a good source of sinapic acid.

Plants may be genetically modified or modified through selective breeding methods to increase the content of sinapine or sinapic acid, for example in the seed of Cruciferous plants of the Brassica family (canola, mustard, etc.). In this way, the invention is a departure from the prior art teachings that work to modify plants to decrease sinapine content (or other phenolics) because of perceived anti-nutritive effects. According to the invention, plants modified to contain extra sinapic acid (or sinapine, in which case the plant material can be treated to form sinapic acid) may be used as a source of isolated sinapic acid, or may be treated and used in a non-purified form simply as a supplement for a feedproduct. Any acceptable method of genetic or selective breeding modifications in plants may be employed to derive a plant that is high in sinapic acid or sinapine, and which is further processed for use in the invention.

Derivatives. Sinapic acid and its derivatives obtained from any source may be used with the invention. Such derivatives for example may be any nutritionally acceptable form of sinapic acid, which would be known to those of skill in the art, and which has the same beneficial effects.

Derivatives of sinapic acid that may be supplemented into the diet according to the invention include salts with inorganic acids, such as hydrochloride, hydrobromide, sulfate and phosphate; salts with organic acids, such as acetate, maleate, tartrate, methanesulfonate; salts with amino acids, such as arginine, aspartic acid and glutamic acid; and salts with bases such as sodium hydroxide and potassium hydroxide. Further, esters of sinapic acid may be used, such as sinapic acid esterified with C1 to C4 groups. As used herein, the ester derivatives include, for example, methyl, ethyl, propyl, or isobutyl sinapic acid.

Sinapic acid and its derivatives can be derived from ester (sinapine), aldehyde (sinapaldehyde) or alcohol (sinapyl alcohol) forms of sinapic acid.

Supplementation Levels. Sinapic acid and its derivatives may be supplemented into feed at levels ranging from about 0.0005% to about 3.0% sinapic acid by weight. An exemplary level of about 0.025% to about 0.2% of feed by weight may be supplemented in the diets of broiler chicks according to the invention.

The above-described embodiments of the present invention are intended to be examples only. Alterations, modifications and variations may be effected to the particular embodiments by those of skill in the art without departing from the scope of the invention, which is defined solely by the claims appended hereto.

Short Chain Fatty Acid Reduction in Hind Gut. Supplementation of feed with sinapic acid has illustrated antimicrobial activity in the digestive tract of broiler chickens and thus provides a natural alternative to antibiotics either as a food or feedstuff preservative or primarily for growth promotion in animals. Dietary sinapic acid caused a large decrease in the total short chain (volatile) fatty acid content (especially acetic acid) production in the hind gut (ceca) of broiler chicks. The observed reduction of short chain fatty acid content by about 10% to 30% was dose dependent (when tested at levels of 0.025%, 0.05%, and 0.10% by weight) indicating strong antibacterial activity in vivo or as well as an ability to modulate fermentation and microflora in the hind gut.

As a possible advantage of sinapic acid supplementation, reduction and/or control of cecal fermentation could benefit poultry or other livestock production by affecting the nature and amount of cecal droppings produced by an animal. This may improve litter conditions in the barn. With respect to egg-laying poultry, may reduce the occurrence of dirty eggs, or eggs exposed to harmful intestinal pathogenic microbes.

Beneficial Microbial Ecology. A reduction of hind gut fermentation was observed, and a shift in microbial ecology was also observed. Antibacterial effects against undesirable microbial populations such as E. coli, S. aureus, and S. enteritidis are also benefits of the invention.

In human health and nutrition, supplementation with sinapic acid will have beneficial effects on intestinal microbial populations, and thus can be used as a micro-ecological modulator in the digestive tract of humans.

In the instant invention, a significant reduction of cecal short chain fatty acid concentrations in all dietary sinapic acid levels supplemented illustrates a growth promoting effect through reduction in total microbial numbers and/or the change in microfloral composition in the lower gut. Sinapic acid can be used as a gastrointestinal microbial modulator that modifies the gastrointestinal fermentation pattern and affects microbial ecology. For some animal and human digestive tract related diseases, such as those caused by the ingestion of more soluble fiber or non-starch polysaccharides ingredients in non-ruminants and acidosis in ruminant animals, the anti-nutritive effects are usually related to excessive fermentation of carbohydrates. Sinapic acid supplementation can be used to prevent these digestive tract diseases, through the modulation on gastrointestinal bacterial fermentation, inhibition on pathogenic bacteria growth, and facilitation of nutrient digestion and utilization. Sinapic acid can be used at prophylactic levels to modulate the microbiological parameters and to improve the nutrient digestion and absorption process in the gastrointestinal tract of animals.

The beneficial effect of sinapic acid and derivatives on microbial ecology of the gut renders sinapic acid to be of use as a therapeutic agent for veterinary or human use in treatment or prevention of disease in the intestinal tract, specifically wherein the disease of the intestinal tract is related to the growth or colonization of the intestinal tract by pathogenic bacteria. A method of treating or preventing such diseases in monogastric mammals by administering sinapic acid or derivatives thereof to the animal is within the scope of the invention. The effective amount of sinapic acid or derivatives thereof can easily be determined by a person skilled in the art by observing the amount required to effect the intestinal microbial ecology. As with feed supplementation, the optimum level of administration is comparable to the amount of 0.0005-3.0% of feed consumption by weight.

When used as a therapeutic agent or as a nutritional supplement, sinapic acid or a derivative thereof can be combined with any generally acceptable excipient, such as a diluent or other non-medicinal compounds as would be known to those skilled in the art.

Effect on Animal Growth and Energy Utilization. Sinapic acid did not affect feed intake in broiler chickens in general. However, feed consumption increased slightly when animals were supplemented at a dose of 0.025% by weight of feed. Sinapic acid improved energy utilization (as determined by measurement of apparent metabolizable energy or AME), and improved fecal protein digestibility. These indices are all beneficial effects for animal performance. Instead of exhibiting anti-nutritional effects, as was thought to be the conventional problem with phenolic food components, sinapic acid actually demonstrated beneficial nutritional effects. Further examples relating to beneficial effects on animal growth and energy utilization are provided below.

Feed-Grade Preservative. When added to plant material, animal feed, or a food product intended for human consumption, sinapic acid and its derivatives have the added benefit of providing preservative effects, and thus acting as a food-grade or feed-grade preservative. This is particularly beneficial for preventing oxidative deterioration of fat-containing ingredients. Additionally, sinapic acid and its derivatives help prevent microbial spoilage or deterioration through antimicrobial effects.

### EXAMPLE 1

### Nutritional, Physiological and Metabolic Effects of Dietary Sinapic Acid Supplementation in Broiler Chickens

Experiments were undertaken to determine the effect of sinapic acid supplementation of the diet of broiler chickens on such parameters as performance, nutrient digestibility, and toxicity.

Materials and Methods. Four treatments were based on a corn-soybean meal diet with or without graded levels of dietary sinapic acid (0, 0.025, 0.05, and 0.10%). Male broiler chicks (Peterson X Hubbard) were randomly assigned into replication groups containing six birds each, and four replications were used for each treatment.

Bird management. Broilers were housed in battery brooders. Temperature was maintained in accordance with standard brooding management and light was provided for 23 h and 16 h from 0 to 5 and 5 to 18 d of age, respectively. Feed, in mash form, and water were provided ad libitum. Sinapic acid was purchased from Sigma™ Chemical Co. (P.O. Box 14508 St. Louis, Mo. 63178 USA) with a purity of 98% (GC grade, Lot 128H3485, light yellow or milky color, dry powder) and was diluted with corn prior to feed mixing. Diets were formulated to be isoenergetic and isonitrogenous, and either meet or exceed the nutrient requirements of broiler chicks as recommended by the National Research Council.

TABLE 1 shows diet composition and nutrient levels.

TABLE 1
Diet Composition and Nutrient Levels^{1,2}

| DIET COMPOSITION | (%) | Nutrient Level | (%) |
|---|---|---|---|
| Corn | 56.61 | AME (kcal/g) | 3.10 |
| Soybean meal | 34.62 | Crude protein | 21.00 |
| Canola oil | 2.82 | Calcium | 0.95 |
| Dicalcium phosphate | 1.56 | Non-phytate P | 0.45 |
| Limestone | 1.60 | Linoleic acid | 1.77 |
| Sodium chloride | 0.46 | Arginine | 1.38 |
| Choline chloride | 0.10 | Lysine | 1.20 |
| Vit./Min. premix² | 0.50 | Methionine | 0.56 |
| DL-Methionine | 0.24 | Methionine + cystine | 0.90 |
| L-Lysine HCl | — | Threonine | 0.81 |
| Celite | 1.50 | Tryptophan | 0.26 |

¹Sinapic acid (98%) was added at 0, 0.025, 0.05, and 0.10%.
²Vitamin and mineral premix supplied per kilogram of diet: vitamin A (retinyl acetate + retinyl palmitate), 11000 IU; vitamin D₃, 2200 IU; vitamin E (dl-α-tocopheryl acetate), 300 IU; menadione, 2.0 mg; thiamine, 1.5 mg; riboflavin, 6.0 mg; niacin, 60 mg; pyridoxine, 4 mg; vitamin B₁₂, 0.02 mg; pantothenic acid, 10.0 mg; folic acid, 0.6 mg; and biotin, 0.15 mg; ethoxyquin, 0.625 mg; iron, 80 mg; zinc, 80 mg; manganese, 80 mg; copper, 10 mg; iodine, 0.8 mg;and selenium, 0.3 mg; calcium carbonate, 500 mg.

Data Collection. Production parameters were monitored from 0 to 18 d of age. Excreta were collected for each pen from 16 to 18 d of age. At the end of 18 d, the birds were individually weighed and killed by chemical agent (T-61™ euthanasia solution), and internal organs and intestines (duodenum, jejunum, ileum and ceca) were removed and measured. For each section of the digestive tract, both full and empty weights were measured. Ileal and cecal digesta were collected from each bird and pooled together within pen, and immediately frozen at −20° C. until further analysis.

Sample Analyses. Acid insoluble ash (AIA, Celite) was used as an indigestible marker for the determination of the apparent digestibility of metabolizable energy, protein (ileal and fecal) and SA. AIA was determined by using the procedure of Vogtmann et al, 1975 Br. Poult. Sci. 67:641-646. The gross energy was measured by a traditional bomb calorimeter, and crude protein was analyzed by a Leco FP-528™ protein analyzer (Model No. 601-500-100, Serial#3211, LECO Corporation, 3000 Lakeview Avenue, St. Joseph Mich. 49085-2396 USA). Based on the assay of nutrient and AIA in feed, excreta, and ileal content, the nutrient digestibility was determined.

The analysis of sinapic acid in feed, excreta and digesta samples was conducted by HPLC using a reversed-phase column under the fluorescence detection method. The mobile phase was isocratic, based on 6% MeOH solution with 20 mmol K₂HPO₄ as basic buffer. For feed and excreta samples, the analysis was conducted at pH 9.5, however, for ileal and cecal samples, the pH was changed to 9 to obtain better background separation and chromatograms.

Statistical Analyses. Data for tissue weights and/or lengths were based on bird weight value/body weight. All data were subjected to one-way Analysis of Variance (ANOVA) according to the General Linear Model (GLM) procedure and a priori contrast, as well regression analysis by the SAS program (SAS™ Institute, 1999). Differences were considered significant when P<0.05, unless otherwise stated.

Results. Supplementation with sinapic acid had effects on animal performance, as evaluated using a number of parameters.

TABLE 2 illustrates results of sinapic acid supplementation on the performance, relative internal organ weight and intestinal measurement, and apparent nutrient and sinapic acid digestibility in broiler chickens.

TABLE 2
Sinapic Acid Supplementation Effects in Broiler Chickens

| | Sinapic Acid Level | | | | | 1 vs | Regression | |
|---|---|---|---|---|---|---|---|---|
| | 0% | 0.025% | 0.05% | 0.10% | SEM | 2-4 | Lin | Quad |
| Gain (g) | 522 | 561 | 545 | 491 | 11.2 | ns | ns | 0.03 |
| Feed intake/bird (g) | 718^{b} | 781^{a} | 738^{ab} | 689^{b} | 11.8 | ns | 0.13 | 0.01 |
| Gain/feed | 0.73 | 0.72 | 0.74 | 0.71 | 0.01 | ns | ns | ns |
| Bursa (g/kg) | 2.32 | 2.29 | 2.46 | 2.29 | 0.09 | ns | ns | ns |
| Kidney (g/kg) | 10.4 | 10.0 | 10.5 | 10.2 | 0.15 | ns | ns | ns |
| Liver (g/kg) | 35.4 | 34.5 | 33.6 | 34.2 | 0.56 | ns | ns | ns |
| Ileum L. (cm/kg) | 93.4 | 84.2 | 92.0 | 94.5 | 1.90 | ns | ns | ns |
| Ceca L. (cm/kg) | 38.9 | 40.6 | 41.0 | 40.4 | 0.57 | ns | ns | ns |
| Ileum F. (g/kg) | 25.7 | 24.6 | 27.0 | 24.5 | 0.65 | ns | ns | ns |
| Ceca F. (g/kg) | 9.8 | 10.1 | 11.2 | 9.5 | 0.43 | ns | ns | ns |
| Ileum E. (g/kg) | 14.5 | 14.1 | 14.7 | 14.4 | 0.18 | ns | ns | ns |
| Ceca E. (g/kg) | 6.1 | 6.2 | 5.9 | 5.4 | 0.14 | ns | 0.04 | ns |
| AME (kcal/kg) | 3348 | 3412 | 3418 | 3442 | 17.4 | 0.27 | 0.07 | 0.08 |
| Ileal protein digest. % | 0.825 | 0.815 | 0.788 | 0.800 | 0.006 | 0.16 | 0.09 | 0.07 |
| Fecal protein digest. % | 0.648 | 0.685 | 0.663 | 0.665 | 0.007 | 0.31 | 0.16 | ns |
| Ileal | — | 0.970 | 0.970 | 0.978 | 0.002 | 0.14 | — | — |
| sinapic acid digest. % | | | | | | | | |
| Fecal | — | 0.793^{a} | 0.638^{c} | 0.713^{b} | 0.020 | 0.0001 | — | — |
| sinapic acid digest. % | | | | | | | | |

^{a,b,c}Values with different letters of superscript in the same row are significantly different (P < 0.05).
SEM—standard error of means,
L—length,
F—full weight,
E—empty weight,
digest.—digestibility.

Body weight gain responded in a quadratic manner to increasing levels of dietary sinapic acid (GLM, P=0.12; quadratic regression, P=0.03) as did feed consumption (quadratic regression, P<0.01). TABLE 2 illustrates these data. Both weight gain and feed intake were highest at the lowest sinapic acid level of 0.025% and declined to near control values for the highest level of sinapic acid inclusion at 0.10%. Feed efficiency was not affected by treatment.

There was no difference among treatments in the relative weight of the bursa of Fabricicus, kidney and liver, and the relative weight and length of the full and empty ileum (Table 2). Cecal length and full weight were not affected by dietary sinapic acid but empty cecal weight decreased with increasing levels of sinapic acid (linear regression, P=0.04).

Apparent metabolizable energy (AME) at all sinapic acid levels were numerically higher than control and regression analysis indicated a linear increase with increasing sinapic acid level, with a significance of P=0.08 (TABLE 2). The effect of sinapic acid on ileal protein digestibility also indicated linear decrease with increasing sinapic acid level (P=0.07). Fecal protein digestibility followed the same pattern as AME with dietary sinapic acid treatments. The numerically highest fecal protein digestibility was found in treatment of the lowest sinapic acid level at 0.025%.

A high proportion of sinapic acid disappeared prior to the ileum as shown by apparent ileal digestibility values between 97.0 and 97.8% for the three sinapic acid diets (Table 2). Significant differences were observed for fecal sinapic acid digestibility among dietary sinapic acid treatments (P<0.001). The lowest level of sinapic acid at 0.025% had the highest value for fecal digestibility. Values for all treatments ranged from 63.8 to 79.3%. There was no sinapic acid found in the cecal digesta.

From these data, it is dear that even a moderate level of supplemental dietary sinapic acid, for example at a level of 0.025% can stimulate feed intake, and improve performance in monogastric animals. Fecal protein digestibility was also improved, illustrating effects in the hind gut.

### EXAMPLE 2

### Sinapic Acid Supplementation in Broiler Chickens (0.05-0.20% Levels)

Eighty day-old commercial broiler cockerel chicks (Peterson X Hubbard) were fed five diets based on corn-soybean meal with one diet free of sinapic acid as the control, and another four diets containing graded levels of sinapic acid (0.05, 0.10, 0.15 and 0.20%) which were equivalent to the sinapic acid profiles of the sinapine moiety in diets containing 7.5, 15.0, 22.5, and 30.0% rapeseed meal. Sinapic acid was purchased from Sigma Chemical Co. (P.O. Box 14508 St. Louis, Mo. 63178 USA). Bird management and diet composition were as described in Example 1.

Volatile Fatty Acid (VFA) Measurement. The status of bacterial populations in the ileum and ceca of experimental birds was assessed by examining VFA production. The birds were killed by lethal injection with T-61™(Euthanasia solution) and the ileal and cecal digesta of three birds within each replication were collected in a well-sealed plastic centrifuge tube and then frozen at −20° C. Ileal digesta were collected from the terminal ileum (distal half excluding the last 2 cm anterior from the ceca). Samples within a replication were pooled and mixed well prior to VFA measurement.

Analyses of VFA (acetic, propionic, isobutyric, butyric, isovaleric, and valeric acid) were conducted by Gas Chromatography (GC) (Varian Star 3400) using a Stabilwax™-DA column (0.25 mm ID, 0.25 μm df) (RESTEK Corporation, 110 Benner Circle, Bellefonte Pa., 16823-8812 USA) and based on the procedure of Choct et al. (Br. Poult. Sci. 1996; 37:609-621) with minor modification which included the use of a pure volatile fatty acid (4-methyl valeric acid in 10% formic acid) that is not normally found in intestinal contents as an internal standard. A concentration of 2 mmol/L is acceptable for the internal standard in intestinal content samples. One ml of standard solution was added to 0.2 (cecal) or 0.5 (ileal) g of sample and vortexed until well-mixed. The mixture was then centrifuged at 28,000 g for 10 minutes and the supernatant pipetted into GC vials for analysis.

Statistical Analyses. All data collected were subjected to one-way Analysis of Variance (ANOVA) using the General Linear Model (GLM) procedure with a priori contrast option, as well as regression analysis (SAS™ Institute, 1999).

Results. In the ileum, regression analysis supported a positive linear relationship between dietary sinapic acid level and ileal levels of acetic, butyric and isovaleric acids, and total VFA. Ileal propionic acid levels were unaffected by sinapic acid level. No valeric acid or isobutyric acid was detected in the ileum digesta of broiler chicks.

Inclusion of sinapic acid in the diet caused a significant quadratic reduction in the level of acetic acid and total VFA in cecal contents. In general, the levels of acetic acid and total VFA were reduced by 15-20% compared with control. Dietary treatment did not affect the level of propionic, butyric, valeric, isovaleric, and isobutyric acid in the cecal contents.

TABLE 3 illustrates that in contrast to the short chain volatile fatty acid production in ceca, dietary sinapic acid increased short chain fatty acid contents in the ileal digesta as sinapic acid levels increased.

TABLE 3
The Effect of Dietary Sinapic Acid on Volatile Fatty Acid Content (mmol/g wet)
of Ileal and Cecal Digesta from Broiler Chickens

| Treatment | Sinapic Acid Level | | | | | | 1 vs | Regression | |
|---|---|---|---|---|---|---|---|---|---|
| VFA | 0% | 0.05% | 0.10% | 0.15% | 0.20% | SEM | 2-5 | Lin | Quad |
| Ileal | | | | | | | | | |
| Acetic | 9.07 | 9.12 | 9.32 | 12.01 | 11.26 | 0.50 | ns | 0.04 | ns |
| Propionic | 0.528 | 0.438 | 0.508 | 0.528 | 0.503 | 0.028 | ns | ns | ns |
| Butyric | 0.090 | 0.098 | 0.093 | 0.113 | 0.130 | 0.006 | ns | 0.01 | ns |
| Iso-Valeric | 0.121^{ab} | 0.061^{b} | 0.118^{ab} | 0.151^{a} | 0.157^{a} | 0.011 | ns | 0.03 | us |
| Total | 9.81 | 9.72 | 10.04 | 12.80 | 12.06 | 0.519 | ns | 0.04 | ns |
| Cecal | | | | | | | | | |
| Acetic | 70.4^{a} | 52.0^{b} | 51.2^{b} | 52.1^{b} | 53.2^{b} | 2.26 | .0006 | 0.01 | 0.01 |
| Propionic | 3.29 | 4.14 | 3.20 | 4.00 | 4.96 | 0.30 | ns | 0.14 | ns |
| Butyric | 14.7 | 15.6 | 16.4 | 17.7 | 15.2 | 0.59 | ns | ns | ns |
| Iso-Butyric | 0.308 | 0.310 | 0.143 | 0.370 | 0.335 | 0.05 | ns | ns | ns |
| Valeric | 0.94 | 0.79 | 0.85 | 1.22 | 1.17 | 0.08 | ns | 0.12 | ns |
| Iso-Valeric | 0.583 | 0.580 | 0.443 | 0.675 | 0.548 | 0.04 | ns | ns | ns |
| Total | 90.2 | 73.4 | 72.2 | 76.1 | 75.5 | 2.52 | 0.01 | 0.11 | 0.06 |

^{a,b}Within a row, values with different alphabet superscripts are significantly different (P < 0.05).
SEM indicates standard error of mean.

The observed increase in total volatile fatty acid production in the ileum in response to dietary sinapic acid corresponded with an increase in the apparent metabolizable energy (AME) of the diet, illustrating that increased short chain fatty acid production in ileum favors more short chain fatty acid absorption in small intestine and improve gut conditions so as to result in more efficient energy utilization. Sinapic acid did not negatively affect feed intake in broiler chickens.

Reduced fermentation in the lower gut is considered to be beneficial in that it represents a suppression of microflora populations, which includes non-beneficial types of microbes. Increasing fermentation in the ileum is associated, in the data obtained, with increased quantities of beneficial bacterial populations. Increased short chain fatty acid in the small intestine (ileum) can be beneficial to animal performance.

### EXAMPLE 3

### Sinapic Acid Supplementation in Broiler Chickens (0.025-0.10% Levels): Effects on VFA Production and Microbial Community

Broiler chickens were fed a corn-soybean meal based diet either unsupplemented or supplemented with sinapic acid at a level of 0.025%, 0.05% or 0.1%. After the supplementation period, microbial analysis was conducted. This Example was designed to confirm the results of Example 2, to assess effects on microbial community, and to repeat the VFA measurement by increasing the chick numbers within the replication.

Ninety-six (120 day-old) male broiler chicks (Peterson X Hubbard) were randomly assigned to four treatments, with six birds in each replication and four replications for each treatment. A corn-soybean based diet served as a control, while another three diets were supplemented with graded levels of sinapic acid (0.025, 0.05 and 0.10%). Bird management was as previously described in Example 1. Volatile fatty acid measurement was conducted as described in Example 2, except that digesta was pooled from six birds per replication.

Microbial Community Assay. An assessment of the microbial community in cecal samples was conducted by Danisco Cultor Corporation, (FIN-02460, Kantvik, Finland). Samples were analyzed according to the method of Apajalahti et al (Appl. Env. Microbiol., 1998;64(10):4084-4088). Cecal digesta samples from 2 birds out of 6 birds within each replication were pooled. Bacterial cells were isolated from the digesta by a five-cycle differential centrifugation process with sodium phosphate buffer. Bacterial cells were lysed by enzymatic incubation with lysozyme followed by SDS incubation with mechanical agitation with glass beads. Samples were then sequentially extracted with CTAB (hexadecyltrimethyl ammonium bromide) and chloroform/isoamyl alcohol prior to alcohol precipitation of the nucleic acids. To obtain a profile of cecal digesta bacterial communities based on by percentage-guanine-plus cytosine (% G+C) content, each DNA sample was subjected to cesium chloride-bisbenzimidazole gradient analysis and centrifuged in an ultracentrifuge. In this analysis, DNA quantitation was based on the UV absorbance at A280. Determination of the % G+C content represented by each gradient fraction was accomplished by regression analysis of data obtained from gradients containing standard DNA samples of known % G+C composition (Clostridium perfringens, Escherichia coli, and Micrococcus lysodeikticus).

Results. Sinapic acid increased feed consumption when supplemented at a dose of 0.025%. This resulted in a 6% increase in weight gain and a 2% increase in diet AME. The stimulation on the feed intake and improved nutrient utilization was found to be dose dependent.

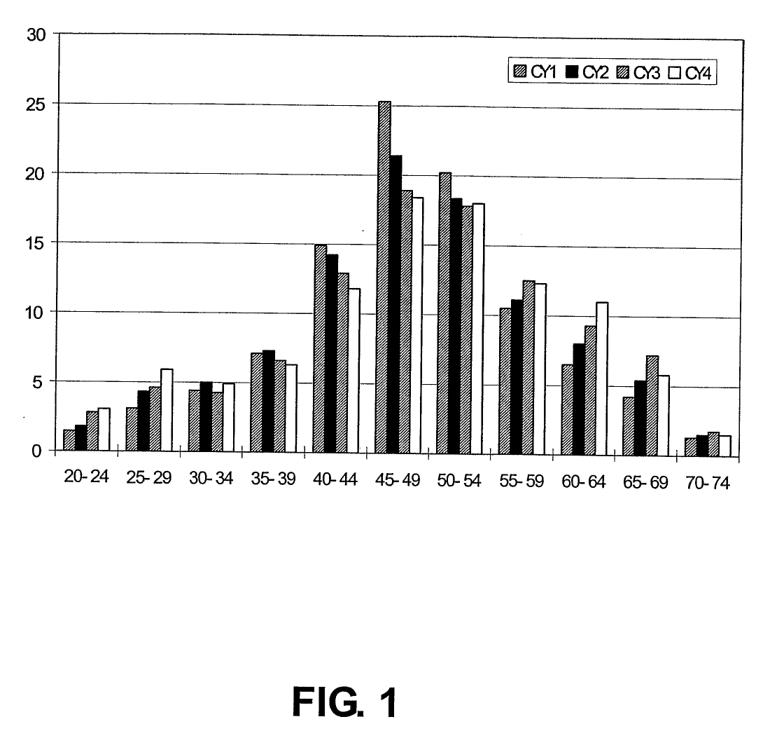

FIG. 1 illustrates microbial analysis of samples using the percentage-guanine-plus-cytosine content in the DNA demonstrated that dietary sinapic acid altered the relative abundance of bacteria in the ceca of broiler chickens. Percentage G+C profiles of cecal microbial community and relative abundance of bacteria in different ranges of % G+C (CY 1: control, CY 2: sinapic acid 0.025%, CY 3: sinapic acid 0.05%, CY 4: sinapic acid 0.10%).

Sinapic acid increased the relative abundance of bacteria in the ranges of % G+C 20-30 and 55-69, and decreased the amounts of microbes in the range of % G+C 40-54 in a dose dependent manner. The shift was more significant as the dosage of sinapic acid given to the broilers was increased. These data illustrate that dietary sinapic acid increased the relative abundance of a small portion of Clostridium in the range from 20-30, and Bifidobacterium and Propionibacterium from 55-69. In contrast, sinapic acid decreased the relative abundance of Escherichia, Salmonella, partial Bacteroides, Eubacterium and Lactobacillus in the range from 40-54, which are the most abundant bacterial genera present in the GI tract of the chicken.

These changes, in general, represent an increase in the relative abundance of bacteria generally considered beneficial, such as Bifidobacterium and Propionibacterium, and a decrease for potentially undesirable bacteria, such as Escherichia and Salmonella. Thus this shift is beneficial to the microbiological ecology in the intestinal tract of an animal, and in turn nutritionally beneficial. It is well known that E. coil and Salmonella are common pathogens in animal production. Probiotics derived from Bifidobacterium and Lactobacillus, are frequently used as competitive exclusion microbial agents against these pathogens and are used as animal health promotants. Sinapic acid can increase animal productivity via increased animal health status. From this standpoint, sinapic acid is an alternative to probiotics and antibiotics normally used as growth and health promotants in animal production.

TABLE 4 illustrates that in contrast to the short chain volatile fatty acid production in ceca, dietary sinapic acid increased short chain fatty acid contents in the ileal digesta as sinapic acid levels increased.

The observed increase in total volatile fatty acid production in the ileum in response to dietary sinapic acid corresponded with an increase in the apparent metabolizable energy (AME) of the diet, illustrating that increased short chain fatty acid production in ileum favors more short chain fatty acid absorption in small intestine and improves gut conditions so as to result in more effident energy utilization. Sinapic acid did not negatively affect feed intake in broiler chickens, and actually increased feed consumption when supplemented at a dose of 0.025%. This resulted in a 6% increase in weight gain and a 2% increase in diet AME. The stimulation on the feed intake and improved nutrient utilization was found to be dose dependent.

Reduced fermentation in the lower gut is considered to be beneficial in that it represents a suppression of microflora populations, which includes non-beneficial types of microbes. Increasing fermentation in the ileum is associated, in the data obtained, with increased quantities of beneficial bacterial populations. Increased short chain fatty acid in the small intestine (ileum) is beneficial to animal performance.

TABLE 4
The Effect of Dietary Sinapic Acid on Volatile Fatty Acid content (mmol/g
wet) of Ileal and Cecal Digesta from Broiler Chickens

| Treatment | Sinapic Acid Level | | | | | Contrast | Regression | |
|---|---|---|---|---|---|---|---|---|
| VFA | 0% | 0.025% | 0.05% | 0.10% | SEM | 1 vs 2-5 | Linear | Quadratic |
| Ileal | | | | | | | | |
| Acetic | 5.52^{b} | 8.32^{a} | 9.14^{a} | 8.39^{a} | 0.42 | 0.0002 | .001 | .004 |
| Propionic | 0.35 | 0.31 | 0.28 | 0.34 | 0.035 | ns | ns | ns |
| Iso-Valeric | 0.058 | 0.055 | 0.013 | 0.055 | 0.009 | ns | ns | ns |
| Total | 5.96^{b} | 8.69^{a} | 9.43^{a} | 8.81^{a} | 0.43 | 0.0007 | .002 | 0.01 |
| Cecal | | | | | | | | |
| Acetic | 106.9^{a} | 93.2^{a} | 74.3^{b} | 68.2^{b} | 4.73 | 0.001 | .0001 | ns |
| Propionic | 6.00 | 4.15 | 4.29 | 4.34 | 0.41 | ns | ns | ns |
| Butyric | 18.1 | 19.9 | 16.3 | 16.7 | 1.00 | ns | ns | ns |
| Iso-Butyric | 0.503 | 0.248 | 0.333 | 0.318 | 0.04 | 0.04 | ns | ns |
| Valeric | 1.60 | 1.44 | 1.28 | 1.06 | 0.09 | 0.08 | 0.02 | ns |
| Iso-Valeric | 3.95 | 3.34 | 2.58 | 0.93 | 0.45 | 0.08 | 0.01 | ns |
| Total | 137.0^{a} | 122.3^{a} | 99.1^{b} | 91.6^{b} | 5.74 | 0.003 | .0003 | ns |

^{a,b}Within a row, values with different alphabet superscripts are significantly different (P < 0.05).
SEM indicates standard error of mean.

This example illustrates that supplemental dietary sinapic acid in rapeseed meal affects volatile fatty acid production (fermentation) and the microbial ecology in the gut of broiler chickens. Dietary sinapic acid consistently increases the short chain fatty acid (VFA) level in the ileum and decreased VFA levels in the ceca. The predominant reduction of VFA in the ceca indicates that dietary sinapic acid can exert antibacterial activity in vivo.

### COMPARATIVE EXAMPLE 1

### Effects of Sinapine Supplementation on VFA Production in Broiler Chickens

To determine whether the effect on VFA content of the ileal and cecal digesta induced by sinapic acid was specific to the sinapic acid form itself, experiments were undertaken for comparative supplementation of animal diets with sinapine.

A complete randomized design was used in which 120 day-old male broiler chickens (Peterson X Hubbard) were randomly assigned to ten treatments, with four replications per treatment and three birds per replication. Treatments consisted of a corn-soybean meal based diet as a control and the same diet supplemented with graded levels (0.15, 0.225, and 0.30%) of sinapine in purified (sinapine bisulfate trihydrate) or semi-purified (ethanol extract concentrate) form. The base diet is the same as that described in Example 2. Three other treatments contained 15, 22.5 and 30% rapeseed meal, which resulted in a sinapine content equivalent to that of the purified and semi-purified diet treatments. The rapeseed meal used in the diet formulations naturally contained 1% sinapine as analyzed.

TABLE 5 illustrates results obtained in this Comparative Example. Dietary treatment with sinapine (in any form) had little impact on total ileal or cecal digesta short chain fatty acid content, relative to the results obtained with dietary sinapic acid supplementation, as shown in Examples 2 and 3.

TABLE 5
The Effect of Dietary Sinapine on Volatile Fatty Acid
content (mmol/g wet) of Ileal and Cecal Digesta from Broiler Chickens

| VFA | Control | | | |
|---|---|---|---|---|
| Content | 0% | 0.15% | 0.225% | 0.30% |
| | | Purified Sinapine (sinapine bisulfate trihydrate) | | |
| Total ileal | 10.4 | 12.4 | 13.7 | 8.9 |
| Total cecal | 82.6 | 79.2 | 73.3 | 78.7 |
| | | Semi-Purified Sinapine (ethanol extract | | |
| | | concentrate) | | |
| Total ileal | 10.4 | 12.5 | 10.8 | 11.2 |
| Total cecal | 82.6 | 85.1 | 74.7 | 80.8 |
| | | Supplemental Rapeseed Meal | | |
| Total ileal | 10.4 | 11.5 | 12.9 | 9.4 |
| Total cecal | 82.6 | 83.4 | 89.7 | 80.5 |

Thus, it is clear from these data that trends in digesta VFA content found with dietary sinapine supplementation are not as distinct or significant as the results observed with sinapic acid supplementation, thus illustrating the specific effect of sinapic acid supplementation.

### INDUSTRIAL APPLICABILITY

The invention provides a method by which the microbial ecology and growth of livestock may be improved, and thus will yield benefit to both small and large farming operations. As the invention applies to domesticated animals, benefit to pet owners of having healthier pets can be realized.

### REFERENCES

Hua et al., 1999. Inhibition of aflatoxin biosynthesis by phenolic compounds. Letters in Applied Microbiology. 29: 289-291.

Nowak et al., 1992. Antioxidative and bactericidal properties of phenolic compounds in rapeseeds. Fat Sci. Technol. 94:149-152.

Qiao and Classen, 2001. Nutritional, physiological and metabolic effects of rapeseed meal sinapine in broiler chickens. Poultry Science. 80 (Suppl. 1): 78.

Qiao and Classen, H. L., 2002. Nutritional, physiological, and metabolic effects of rapeseed meal sinapic acid (3,5-dimethoxy-4-hydroxy-cinnamic acid) in broiler chicks. Poultry Science. 81 (Suppl. 1). 26.

Tesaki et al., 1998. 4-Hydroxy-3-nitrophenylacetic and sinapic acids as antibacterial compounds from mustard seeds. Biosci. Biotechnol. Biochem 62 (5):998-1000.

Wanasundara et al., 1994. Isolation and identification of an antioxidative component in canola meal. J. Agric. Food Chem. 42:1285-1290.

1. An animal feed composition for monogastric non-human animals comprising agriculturally acceptable feed components supplemented with sinapic acid or a derivative thereof.

2. The composition of claim 1 wherein sinapic acid or a derivative thereof is in an isolated form.

3. The composition of claim 1 wherein sinapic acid or a derivative thereof is supplemented at a level of about 0.005% to 3% by weight.

4. The composition of claim 3 wherein sinapic acid or a derivative thereof is supplemented at a level of from 0.025% to 0.2% by weight.

5. The composition of claim 1 wherein the derivative of sinapic acid is selected from:
salts of sinapic acid with inorganic acids, such as hydrochloride, hydrobromide, sulfate and phosphate;
salts of sinapic acid with organic acids, such as acetate, maleate, tartrate, methanesulfonate;
salts of sinapic acid with amino acids, such as arginine, aspartic acid and glutamic acid;
salts of sinapic acid with bases such as sodium hydroxide and potassium hydroxide, and
esters of sinapic acid esterified with C1 to C4 groups such as methyl, ethyl, propyl, and isobutyl sinapic acid.

6. A nutritional supplement for a monogastric animals comprising isolated sinapic acid or a derivative thereof in combination with an acceptable excipient.

7. The nutritional supplement of claim 6, wherein the monogastric animal is a non-human animal.

8. A method of promoting favourable microbial ecology in the intestinal tract of a monogastric animal comprising the step of providing the animal with feed supplemented with sinapic acid or a derivative thereof in an amount of from 0.0005% to about 3.0% by weight of feed.

9. The method of claim 8 wherein promoting favourable microbial ecology comprises reducing intestinal presence of at least one microbe selected from the group consisting of Escherichia and Salmonella; or increasing intestinal presence of at least one microbe selected from the group consisting of Bifidobacterium and Propionibacterium.

10. A method of improving the nutritional value of an animal feed composition for consumption by monogastric non-human animals comprising the step of supplementing the feed composition with sinapic acid or a derivative thereof.

11. The method of claim 10 wherein the composition is supplemented with sinapic acid or a derivative thereof at a level of about 0.0005% to about 3% by weight.

12. The method of claim 10 wherein sinapic acid or a derivative thereof is in an isolated form.

13. The method of claim 10 wherein the animal is a bird, pig, dog, cat or fish.

14. The method of claim 10 wherein improving the nutritional value of an animal feed composition comprises increasing the performance of a livestock animal consuming the animal feed composition.

15. The method of claim 14 wherein increasing the performance of a livestock animal comprises improving growth, energy utilization or feed consumption.

16. A method of reducing short chain fatty acid production in the hind gut of a monogastric animal comprising the step of administering an effective amount of sinapic acid or a derivative thereof to the monogastric animal.

17. The method of claim 16 wherein the effective amount is from about 0.0005% to about 3.0% by weight of feed consumption.

18. A method of increasing short chain fatty acid production in the ileum of a monogastric animal comprising the step of administering an effective amount of sinapic acid or a derivative thereof to the monogastric animal.

19. The method of claim 18 wherein the effective amount is from about 0.0005% to about 3.0% by weight of feed consumption.

20. A method of treating or preventing diseases of the intestinal tract arising from growth or colonization of the intestinal tract by pathogenic bacteria, said method comprising the step of administering an effective amount of sinapic acid or a derivative thereof to a monogastric animal in need thereof.

21. The method of claim 20 wherein the effective amount is from about 0.0005% to about 3.0% by weight of feed consumption.
